(19) United States
(12) Patent Application Publication
(10) Pub. No.: US 2023/0285626 A1
(43) Pub. Date: Sep. 14, 2023

(54) TABLE-TOP APPARATUS FOR GENERATING A SUBSTANTIALLY MICROBE-INACTIVATED ROOM ZONE

(71) Applicant: OSRAM GmbH, Muenchen (DE)

(72) Inventor: Markus STANGE, Berlin (DE)

(21) Appl. No.: 18/019,216

(22) PCT Filed: Aug. 30, 2021
(86) PCT No.: PCT/EP2021/073876

§ 371 Date: Feb. 1, 2023

(30) Foreign Application Priority Data
Sep. 21, 2020 (DE) 10 2020 211 777.2

(51) Int. Cl. A61L 9/20; F24F 13/06; F24F 8/22; F24F 3/16

(52) U.S. Cl. CPC A61L 9/20; F24F 13/0604; F24F 8/22; F24F 3/16

(57) ABSTRACT

A table-top apparatus for generating a substantially microbe-inactivated room zone comprises a housing with an interior, which has a longitudinal axis, a blower device designed to receive air from outside and convey it into the interior, a radiation source designed to emit light in the UV-C spectral range into the interior, in order to inactivate or kill off microbes in the received air, and an air outlet device through which the sterilized air can flow from the interior into a space surrounding the housing. The air outlet device is designed to produce a preferably laminar flow in the outflowing air, which supports the formation of the substantially microbe-inactivated room zone within the surrounding space. The substantially microbe-inactivated room zone generated in this way is closed off and completely envelops the housing when the latter is viewed in a plane perpendicular to the longitudinal axis of the housing.

### TECHNICAL FIELD

The invention relates to a table-top apparatus for generating a substantially germ-inactivated spatial zone around one or more persons who is or are spending time in, for example, a public place such as a restaurant together with other persons.

### BACKGROUND

It is known to provide ventilation and air recirculation systems, for instance in buildings, or alternatively air-conditioning installations, with UV-C radiators in order to inactivate or kill germs or pathogens, for instance bacteria, parasites, germs, viruses or viroids, fungi or algae, etc., from the room air. Typically, for this purpose air is taken from the corresponding rooms, is exposed to UV-C radiation during the conditioning, and is finally delivered back to the corresponding room. A wavelength range corresponding to UV-C radiation extends from 100 nm to 280 nm. For example, it is possible to use low-pressure mercury vapor lamps, which emit radiation or light with a wavelength of 254 nm, this being used for example for virus inactivation since in this case the viral nucleic acid is attacked. After multiple cycles, the germ load in the relevant rooms may be reduced by more than 99% using this treatment.

During the pandemic caused in 2020 by the coronavirus SARS-CoV-2, inter alia in Germany and the European Union extensive measures were implemented by which the population was intended to be protected from infection and further spreading. Inter alia, the wearing of mouth/nose protection masks and compliance with minimum distances between persons or maximum numbers of persons in closed spaces or in open-air activities were legislated or at least recommended in order to minimize the germ load in the surrounding air and prevent ingress of germs into the respiratory tracts. The described use of UV-C disinfection in ventilation and air recirculation systems or in air-conditioning installations may certainly make a contribution to this, but no guarantee can be made that air returned through many cycles is substantially germ-inactivated in the general case, that is to say other than in cases of dedicated systems in clean rooms or hospitals. Such systems fulfilling the requirements would be too complicated and too expensive to be usable in practice, for example in restaurants, cafés or other places where people meet and spend some time.

Publication DE 197 42 358 A1 describes a transportable miniature air purification apparatus. The apparatus is configured as a handheld apparatus and has a housing with a blower arranged therein, as well as an air purification stage for generating a purified air flow. The apparatus may be put on a planar surface, and the housing has an inflow grille on one side, through which ambient air is suctioned, and a blowing grille on the other side, through which the disinfected air is blown out again. The purification and disinfection are carried out by ultrafine microfilters, UV lamps and activated carbon filter elements respectively arranged successively in the air flow. With the aid of a laminar flow body at the blowing grille, a laminar air flow is obtained by which differently sized lobe-shaped zones of air with reduced pollution are achieved, depending on the blower setting. The freedom from vortices associated with the laminar clean air flow in this case, in particular, counteracts mixing of the flow with ambient air in its outer regions. A distance of the end of the zone of air with reduced pollution from the air outlet region of the apparatus is in particular between 0.7 and 1.5 m, and the flow rate in the central region of the laminar outlet air flow is not more than 0.5 m/s, in particular less than 0.3 m/s.

Unfortunately, such an apparatus is quite unsuitable for use in restaurants, cafés, etc., for example in the scope of combating viruses. The suction on one side and the blowing on the other side leads to an air movement generally directed horizontally in the room. Since each occupant of the room would need their own apparatus because of the size of the germ-inactivated spatial zone thus generated, this would lead to mutual interference of the air flows partly opposing one another or crossing over. Due to this, a perturbation or instability of the laminar air flows actually intended would be likely, that is to say in particular an at least partial reduction of the turbulence-free spatial zone and therefore an undesired ingress of germ-laden ambient air, which above all is also unnoticed by the user. Furthermore, the users would also need to check regularly whether or not they are currently located in the direction of the flow blown out, and as described, the users would not be capable of identifying contamination as a result of undesired turbulence. The risk therefore arises that the users as well as the managers of restaurants, cafés, etc., may be lulled into a false sense of security.

### Presentation of Various Aspects

In order now to offer a solution to these or similar problems, an improvement is sought according to aspects and exemplary embodiments mentioned below such that a substantially safe and stable as well as substantially germ-inactivated, or in this case germ-inactivated and person-related, spatial zone is simultaneously provided for as many occupants as possible of a public place (restaurant, café etc.), without significant mutual interference of the zones taking place.

According to one exemplary embodiment, a table-top apparatus for generating a substantially germ-inactivated spatial zone is proposed, which has a housing with an interior, a suction duct connected to the housing, a blower device, a UV-C lamp and an air outlet device. Unlike in the prior art, the table-top apparatus does not need to have filters or activated carbon elements, which may advantageously lead to a saving on area and space and therefore to a compact table-top apparatus. Nevertheless, these elements are not however precluded in principle.

The suction duct is used to take air in from the environment outside the spatial zone generated by the apparatus itself and convey it into the interior of the housing. The site of the suction opening may in this case be positioned at a location adapted to the situation, depending on the embodiment, without restriction of generality for example above or below a table on which the table-top apparatus is put. The intake process for the ambient air is driven by the blower device. The blower device may be arranged in the suction duct itself or in the housing, particularly in the interior of the latter. The suction duct extends, starting from the housing, into the surrounding space.

The UV-C lamp is configured to emit light in the UV-C spectral range, that is to say particularly in the wavelength range of from 100 nm to 280 nm, into the interior in order to inactivate or kill germs in the suctioned air. It may in particular be a mercury low-pressure lamp. Other types of lamp, for example quartz lamps emitting in the UV-C range or light-emitting diodes (LEDs), are not, however, precluded. Mercury-based lamps offer an advantage that they can be arranged along a longitudinal axis of the interior and may therefore be fitted per se according to the intended symmetry of the table-top apparatus or even in the interior of the suction channel. In the case of LEDs, this is naturally also possible by using a corresponding arrangement.

The light source may additionally be surrounded in the interior of the apparatus by a UV-transmissive envelope (for example a quartz tube), by which the cool air flow of the source is decoupled from the useful air flow of the inactivated air zone of the apparatus, so as to protect the user from the heat given off by the source.

The air outlet device can direct the flow of the sterilized air out from the interior into a space surrounding the housing. The air outlet device is in this case configured so as to form a substantially laminar, or low-turbulence flow in the air flowing out. This flow generates the substantially germ-inactivated (that is to say significantly germ-reduced) spatial zone inside the surrounding space. In particular, a region with tranquil air, which is substantially free of vortices that contain untreated air, is generated by the flow. This leads to stable separation from the potentially germ-laden air surrounding the germ-inactivated spatial zone. The germ-inactivated spatial zone is consequently closed off from the environment. This does not preclude the possibility that vortices may occur in the substantially germ-inactivated spatial zone. What is important is that they introduce scarcely any germ-laden air into the spatial zone.

One particular feature of the proposal made here is now that the flow is configured in such a way that the housing is fully enclosed by the substantially germ-inactivated spatial zone, and the suction duct coming from the housing during operation extends through the germ-inactivated spatial zone as far as its edge region and beyond the latter, and thereby allows suction from the surrounding air which is not germ-inactivated. The suction duct functions here in a similar way to a snorkel from the spatial zone. Furthermore, the suction duct may be arranged in such a way that it draws in air from a region of the environment which is far enough away from the germ-inactivated spatial zone of the associated table-top apparatus but also far enough away from the germ-inactivated spatial zones of further table-top apparatuses placed in a typical restaurant or café, so that the spatial zones of the latter are not perturbed.

The interior of the housing in this case extends along a longitudinal axis and the enclosure of the housing by the germ-inactivated spatial zone generated by the table-top apparatus is considered in a plane perpendicular to this longitudinal axis. The flow is likewise directed perpendicularly to the longitudinal axis. A width of the germ-inactivated spatial zone in the direction of the longitudinal axis is therefore only insubstantially greater than the longitudinal dimension of the table-top apparatus along this longitudinal axis. According to a further aspect of the invention, a plurality of such table-top apparatuses may be placed next to one another while being aligned along the longitudinal axis, in order to achieve a desired width of the germ-inactivated spatial zone in the direction of the longitudinal axis. A vertical setup of the part blowing out is furthermore conceivable, if enclosure of the persons is thereby still ensured.

Conversely, an extent of the germ-inactivated spatial zone starting from the table-top apparatus, or from the housing of the latter, may be configured so amply, depending on the blower device (or the power delivered by the latter) and the geometry of the air outlet device, that, when the table-top apparatus is placed on a planar surface of a table, for example in a restaurant or café, the head and torso of a person sitting at this table are reliably included in the germ-inactivated spatial zone.

By extending the substantially laminar or low-turbulence flow, with the same table-top apparatus a second person sitting at the table may also be provided with their own germ-inactivated spatial zone on the side of the housing lying directly opposite (in a direction perpendicular to the longitudinal axis). This is possible because the air suction through the suction duct takes place in a region outside the germ-inactivated spatial zone, preferably in a vertical direction above the table-top apparatus when the latter is placed on the table, but also for example from below the table or even from outside the room. The laminar flow, or the laminar flows, are then for example mutually symmetrical so that the germ-inactivated spatial zone stably encloses the table-top apparatus. For example, in this way less air movements in the room, which could for example interfere with germ-inactivated spatial zones generated by further table-top apparatuses, may also be generated.

In this way, it is possible to equip a plurality of tables with the proposed table-top apparatuses in order to generate many person-specific germ-inactivated spatial zones, so that a reduction of the germ load-dependent minimum distance between the persons in this space is even made possible. Furthermore, the individual user may establish the enclosing germ-inactivated spatial zone in which they can have confidence merely by the position of the apparatus on the table, and not necessarily by its alignment. Objective features, which are free from operating error, may therefore be made verifiable in order to ensure the person-specific germ-inactivated spatial zones in a restaurant or a café, etc., in order possibly to reduce the minimum distance or make the need to wear a mask superfluous.

According to one preferred development of the exemplary embodiment of the proposed table-top apparatus, the spatial zone substantially considered to be reliably germ-inactivated has a maximum extent from the housing, and the suction duct has an opening through which the air to be suctioned is taken in. A distance of the opening from the housing is in this case greater than the maximum extent of the germ-inactivated spatial zone as calculated from the housing. The maximum extent refers to a distance between the edge region of the germ-inactivated spatial zone and the housing, or the air outlet device. This relationship ensures that the entry duct takes in air from a region outside the germ-inactivated spatial zone, so that the germ-inactivated spatial zones per se are stably maintained and air is not drawn in a circuit from these themselves.

According to another preferred development of the exemplary embodiment, the distance of the opening of the suction duct from the housing is 80 cm or more, preferably 90 cm or more, more preferably 100 cm or more, when the suction duct protrudes in a direct line from the germ-inactivated zone. With a differently designed separation of the suction opening from the aforementioned zone, for example the tabletop, the suction duct may also be made significantly shorter. Such a distance is, for example, sufficient in order to suction air sufficiently far above the germ-inactivated spatial zone and the heads of the persons located in the spatial zone.

According to another preferred development of the exemplary embodiment, the maximum extent of the substantially germ-inactivated spatial zone as calculated from the housing is 80 cm or less. Preferably, the maximum extent may also be 70 cm or less, or even 60 cm or less. These distances of the edge region of the germ-inactivated spatial zones ensure that the heads of the persons sitting at the table are reliably located inside the germ-inactivated spatial zones.

According to another preferred development of the exemplary embodiment, the air outlet device has a grille structure having a multiplicity of air outlet openings, each of which gives rise to flow vectors in the air flowing out through them, the flow vectors in their entirety covering a full semicircle of at least 180 degrees in planes perpendicular to the longitudinal axis, perpendicularly over a surface on which the table-top apparatus is put during operation. Because the air outlet device emits the air flowing out in a full semicircle, a particularly stable germ-inactivated spatial zone is generated, at the center of which the housing of the table-top apparatus is situated. If 2 persons are sitting opposite at a table, the longitudinal axis of the interior of the housing, or of the table-top apparatus, is preferably oriented perpendicularly to a connecting line between the two persons. In other words, the two persons are located within the aforementioned plane and are thereby reliably included in the corresponding germ-inactivated spatial zones.

According to another preferred development of the exemplary embodiment, the air outlet device in the table-top apparatus is configured in such a way that two laminar flows substantially lying opposite one another are formed perpendicularly to the longitudinal axis, the flow rate thereof in each case being 0.5 m/s or less. This aspect advantageously relates in particular to a situation in which 2 or more persons are sitting opposite one another at the table and each is included in their own part of the germ-inactivated spatial zone.

According to another preferred development, the flow rate is in each case 0.2 m/s or less, preferably about 0.1 m/s. These low values are possible in particular because the germ-inactivated spatial zone is configured as an enclosure for the housing of the table-top apparatus. The user will in this case notice even less of the air flow.

According to another preferred development of the exemplary embodiment, the radiation source is configured to emit radiation in the UV-C spectral range with a dose of 50 J/m² or more, preferably 100 J/m² or more. These values deliver a sufficient dose to ensure the germ inactivation.

According to another preferred development of the exemplary embodiment, the table-top apparatus comprises a reflector device by which the interior is irradiated with the light emitted by the lamp. The reflector device may be a reflector or, in the case of a mercury low-pressure lamp tube, one having a parabolic cross section which extends in the same way as it along the longitudinal axis. This allows particularly effective illumination of the interior and therefore particularly high-quality germ inactivation. Other cross sections which ensure good illumination of the interior, together with shielding of the UV radiation outward, may however likewise readily be envisioned.

According to another preferred development of the exemplary embodiment, the interior is mirrored in order to achieve homogeneous illumination of the interior. According to another preferred development of the exemplary embodiment, at least a part of the irradiated interior is coated with TiO₂ (anatase). This may, for example, prevent subjectively unpleasant odor formation.

According to another preferred development of the exemplary embodiment, the air outlet device has an inner first grille structure or perforated structure (screen), of which the arrangement and sizes of the holes assists the formation of the shape of the aforementioned spatial zone, an outer second grille structure, and an air-permeable membrane arranged between them. Preferably, the outer second grille structure and the air-permeable membrane arranged between the grille structures are in this case configured so as to be mechanically replaceable by using manually releasable fastening means. The membrane in this case protects the apparatus in particular from contamination by droplets possibly containing germs, which are released by users or guests at the table and impinge on the table-top apparatus. It may be a fabric similarly as in simple mouth protection masks or similar nonwoven fabrics or substances. Replaceability ensures that, in the event of the users or guests at the table changing, the disinfected air flowing out does not acquire the germs from the droplets which have reached the fabric from the previous guests.

According to another preferred development of the exemplary embodiment, the table-top apparatus comprises a monitoring unit having a preferably wireless-based communication unit, which is configured to communicate data relating to the function and the operating state of the table-top apparatus to an external control device. The representation of the operating state may also be carried out by means of simple status indicators, for example by an LED or display on the apparatus itself. The communication may be carried out by means of Bluetooth, WLAN/WiFi, NFC etc. Cabled communication is, however, also included in principle. This aspect allows monitoring and control of the table-top apparatus, and also optionally the generation of an alarm if the local germ inactivation is no longer ensured owing to an apparatus fault. It is also possible to establish communication between the table-top apparatus and the cell phones (smartphones) of the respective guests, for example by means of Bluetooth. The users or guests are in this way directly informed of their safety status (that is to say the existence of the germ inactivation of their personal spatial zone).

According to another preferred development of the exemplary embodiment, the table-top apparatus comprises a sensor for recording the dose of the light emitted, the air flow generated or a distance of a person located in the surrounding space from the housing. In this way for example, in cooperation with a control device, a control loop can be set up, by which the germ-inactivated spatial zone is adapted in its extent in such a way that the person in question is reliably included in the germ-inactivated spatial zone while the noise development is reduced further by minimizing the necessary blower output.

According to another preferred development of the exemplary embodiment, the table-top apparatus comprises docking means by which a further table-top apparatus of the same design may be docked to the table-top apparatus along the longitudinal axis, in order to increase the substantially germ-inactivated spatial zone in the direction of the longitudinal axis. This increases the variability of the system envisioned, and also makes it possible to set up larger germ-inactivated spatial zones along longer tables.

Further advantages, features and details of the various aspects may be found in the claims, the following description of preferred embodiments and with the aid of the drawings. In the figures, references which are the same denote features and functions which are the same.

### BRIEF DESCRIPTION OF THE DRAWINGS

FIG. 1 shows a first exemplary embodiment of a table-top apparatus for generating a germ-inactivated spatial zone during operation on a table in a café;

FIG. 2 shows a schematic perspective view of the table-top apparatus of FIG. 1;

FIG. 3 shows a schematic perspective view of the table-top apparatus as in FIG. 2, but with a representation of the inside of the apparatus;

FIG. 4 shows a schematic cross-sectional view of the table-top apparatus of FIG. 2 with a representation of the flow coverage of the surrounding space, the plane of the drawing being a plane perpendicular to the longitudinal axis L of the table-top apparatus;

FIG. 5 shows a second exemplary embodiment of a table-top apparatus for generating a germ-inactivated spatial zone in a schematic cutaway perspective view;

FIG. 6 shows an excerpt of FIG. 5 with more details of the corresponding air outlet device;

FIG. 7 shows a third exemplary embodiment of a table-top apparatus for generating a germ-inactivated spatial zone in a schematic perspective view;

FIG. 8 shows the table-top apparatus of FIG. 7 in a schematic cross-sectional view during operation on a table;

FIG. 9 shows the table-top apparatus of FIG. 7 coupled to further table-top apparatuses of the same design;

FIG. 10 shows a fourth exemplary embodiment of a table-top apparatus for generating a germ-inactivated spatial zone with a suction duct directed vertically upward;

FIG. 11 shows a fifth exemplary embodiment of a table-top apparatus for generating a germ-inactivated spatial zone with a suction duct taken down under a tabletop;

FIG. 12 shows a sixth exemplary embodiment of a table-top apparatus for generating a germ-inactivated spatial zone with lateral droplet protection.

### PREFERRED EMBODIMENTS OF THE INVENTION

In the following description of preferred exemplary embodiments, it should be remembered that the present disclosure of the various aspects is not restricted to the details of the construction and the arrangement of the components as they are presented in the description below and in the figures. The exemplary embodiments may be implemented or carried out in various ways in practice. It should furthermore be remembered that the expressions and terminology employed here are used merely for the purpose of concrete description, and they should not be interpreted by a person skilled in the art as such in a restrictive way.

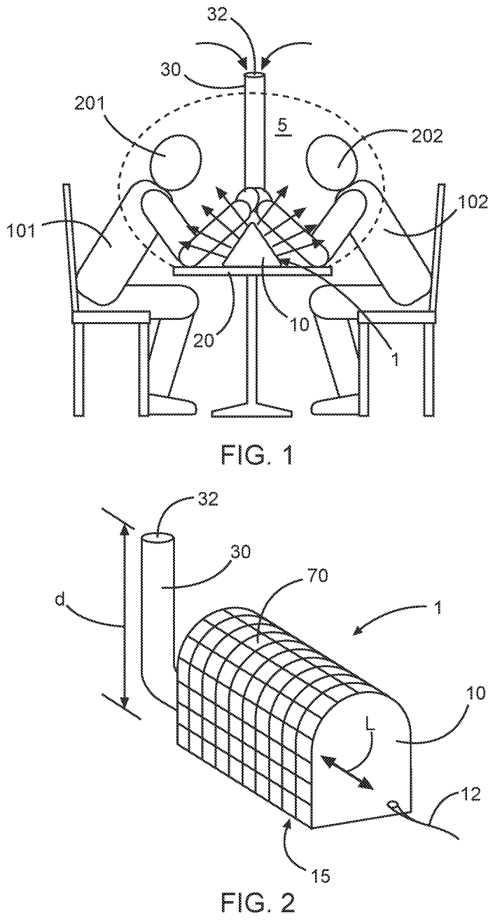

A first exemplary embodiment of a table-top apparatus for generating a germ-inactivated spatial zone will be explained with reference to FIGS. 1 to 4. FIG. 1 illustrates a schematic representation of the operation of a table-top apparatus 1 according to the exemplary embodiment on a table in a restaurant or café, at which two guests 101, 102 have occupied seats opposite one another. In the representation, the table-top apparatus 1 comprises a housing 10 and a suction duct 30, which is connected to the housing 10 and at the end of which facing away from the housing 10 an opening 32 intended for the suction of unsterilized air 120 is arranged. The table 20 may be located in a closed seating area or in an outdoor region of the restaurant or café. By operation of the table-top apparatus 1, inside the restaurant or café a substantially germ-inactivated spatial zone 5 with germ-inactivated air is generated, the extent of which is so great that the heads 201, 202 of the guests 101, 102 involved are reliably included therein when the guests 101, 102 have taken seats at the table 20 on both sides of the table-top apparatus 1.

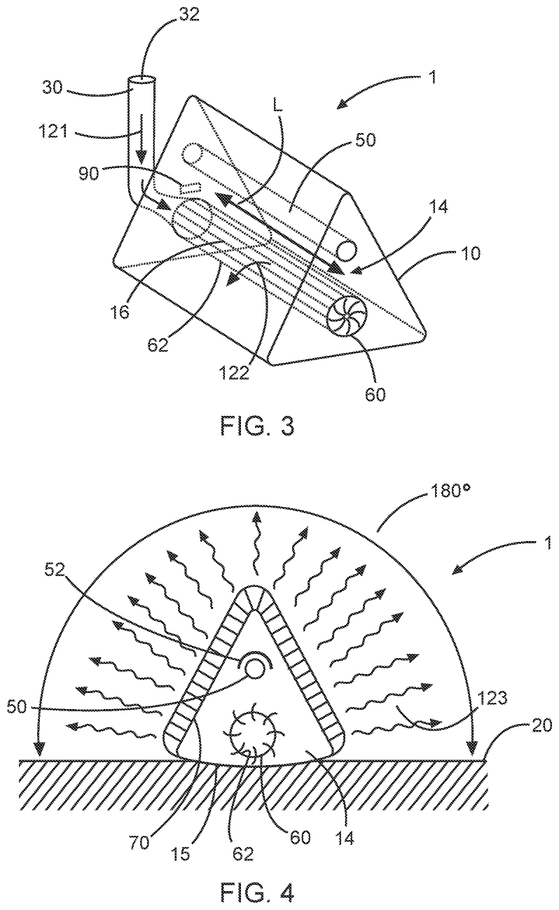

FIG. 2 shows the table-top apparatus 1 in a schematic perspective representation. The housing 10 extends along a longitudinal axis L and has, for example, an inverted “U” or “V” cross section. The housing 10 is closed on the end sides, while the lateral face extending along the longitudinal axis L may be configured as a grille structure or as a pattern of holes, which is used as an air outlet device 70 through which the blower device 60 shown in FIG. 3 makes germ-inactivated air flow out. FIG. 3 shows the table-top apparatus 1 schematically in a transparent representation in order to reveal the basic inner construction of the housing 10.

First, however, the still unsterilized air 120 is taken in from the environment of the spatial zone 5 through the opening 32 of the suction duct 30, which is fitted laterally on the housing 10 (on one of the end sides) and is connected to the interior 14 shown in FIG. 3. Through the suction duct, the air 121 taken in is conveyed into the interior 14, or into an air repository 16 accommodated therein, which in this exemplary embodiment is configured as a tube 62 and extends along the longitudinal axis L. Provided in the air repository 16, or the tube 62, there is the blower device 60 configured as a propeller or fan, which generates a reduced pressure in the air repository 16 and in the suction duct 30 so that air 120 is taken in. Provided over the length of the tube 62, there are openings (not shown in detail in FIG. 3) through which air can emerge into the (rest of the) interior 14 while being homogeneously distributed.

A UV-C radiation source 50 likewise extending along the longitudinal axis L over the length of the interior 14, which may be configured as a mercury low-pressure lamp and irradiates as homogeneously as possible the interior 14 with the air 122 flowing therein (only indicated by an arrow in FIG. 3) is also provided there. A suitable reflector device 52 may additionally be present, such as is indicated for instance in FIG. 4. This reflector device 52 ensures that no UV-C radiation emerges outward through the grille-like air outlet device 70, and furthermore improves the homogeneity of the irradiation of the interior 14. The reflector device 52 may have a parabolic cross section, may be configured as a tube, and may have closed or open polygonal cross sections, in particular a tube having a hexagonal cross section which encloses the mercury low-pressure lamp. The design depends on the shape of the interior 14 to be illuminated.

FIG. 4 represents the way in which the air 123 now disinfected by UV-C radiation flows out through the air outlet device 70, while generating two laminar, or low-turbulence, flows directed obliquely upward to the right and left in FIG. 4 at the heads 201, 202 of the guests 101, 102. An air flow is also released upward, that is to say between the two laminar air flows. The reduced pressure generated in the interior 14 by the blower device 60, the size and number of the air outlet openings in the grille structure or the pattern of holes, and their flow vectors, etc., are matched to one another so that the laminar flows are achieved. Overall, a stable spatial zone which is closed off in respect of air exchange with the environment and is substantially germ-inactivated because of the UV-C radiation is formed above the surface of the table 20 on which the table-top apparatus 1 is put. The flow vectors in this case cover in their entirety a full semicircle of at least 180 degrees in planes perpendicular to the longitudinal axis L, perpendicularly over the surface of the table 20 on which the table-top apparatus is put during operation. The plane of the drawing in FIG. 4 is such a plane perpendicular to the longitudinal axis L.

“Closed off” means here that in respect of the laminar flow streams of the spatial zone 5, their collapse at the outer boundary (formation of fluctuating vortices) forms a boundary which is substantially stationary during operation, and air continuously fed thereto is released to the environment. In the region between the laminar flows, stationary vortices may for example also be formed, which release air only gradually to the environment and are constantly replenished with disinfected air from inside the spatial zone. A characteristic feature is a continuous spatial zone 5 which encloses the table-top apparatus 1 itself because the table-top apparatus 1 releases disinfected air 123 on all sides, the spatial zone 5 having a level of contamination which is significantly lower than that of the environment. Inside the spatial zone 5, the level of contamination is relatively homogeneous and stable. The boundary region marking the difference in contamination level between the inside and the outside is spatially almost stationary. At the location of the persons inhaling the air (heads 201, 202), there is a low germ load due to more than 95%, preferably 99%, more preferably 99.9% inactivation.

The distance d of the opening 32 of the suction duct 30 is, in the particular exemplary embodiment, for example 80 cm. The maximum extent h of the spatial zone (above the table 20) is for example 60 to 70 cm. This distance should be greater than the extent of the zone 5 and the distancing may in this case also be carried out by extending the suction channel 30 with other objects that delimit the zone 5 from the outside air 120, for example the tabletop itself. (In this case, the channel 30 actually assigned to the apparatus may also be significantly shorter.) In this way, the table-top apparatus 1 may reliably take air in from the environment and supply the spatial zone 5 from the inside with newly inactivated air, and therefore replace the air leaving at the outer boundary 6 of the spatial zone 5 (in this regard see also FIG. 8) by fluctuating vortex formation. Spatial stability of the spatial zone 5 is thereby ensured. The shape of the spatial zone is impaired at most by sizeable continuous air movements in the room, for instance when all the windows are open in order to ventilate the room and outside there are prevailing winds which therefore also reach into the space of the restaurant or café.

Furthermore, FIG. 2 also shows a schematic representation of a sensor 90. With the latter, data relating to the function and the operating state of the tabletop may be recorded. This may for example be the dose of the light emitted, the magnitude of the air flow generated or a distance of a person located in the surrounding space from the housing. These data may be communicated to a control device (not shown in the FIGS. which can provide warning indications or initiate the technical measures by which problems may be resolved or limit values being exceeded or fallen below may be compensated for or balanced. For this purpose, a monitoring unit having a preferably wireless-based transmission and reception unit, which is configured to communicate such data relating to the function and the operating state of the table-top apparatus to an external control device, may also be interposed. The monitoring unit and/or the control device may also be implemented on a cell phone (for example as an app), in particular of one or more guests. For example, a warning may be displayed that the operation of the table-top apparatus is currently so impaired that a germ-inactivated spatial zone 5 protecting the guest can no longer reliably be maintained.

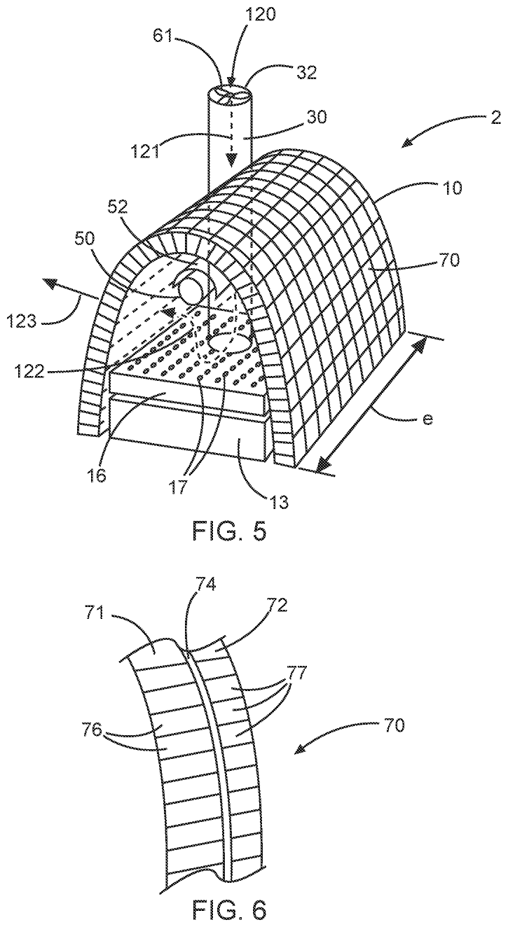

FIGS. 5 and 6 show a second exemplary embodiment of a table-top apparatus 2 as proposed here. The suction duct 30 is constructed similarly as in the first exemplary embodiment, although a corresponding blower device 61 is in this case provided inside this suction duct 30 at the opening 32. Furthermore, the air repository 16 is in this case configured as a flat cuboid space in the lower region of the interior 14.

The suction duct 30 leads through an opening 34 into the air repository 16, where the air 121 taken in is introduced. A multiplicity of holes 17 allow a homogeneous flow of the air 122 in from the air repository 16 upward into the interior 14 for the purpose of disinfection with UV-C irradiation by the radiation source 50 (with a reflector device 52, as above). The air outlet device 70 is shown in more detail in FIG. 6. In the excerpt shown, it comprises an inner grille structure 71 with air outlet openings 76, a membrane 74 which is used as replaceable spittle protection and may be made from a fabric or nonwoven, and an outer grille structure 72 with air outlet openings 77. The outer grille structure 72 is removable and is used to hold the membrane 74 on the inner grille structure 71. The flow vectors of the air outlet openings 76 of the first inner grille structure 71 are relevant for the formation of the laminar air flows, or the substantially germ-inactivated spatial zone at all. In addition or instead, the desired flows may however also be generated equally well by the second outer grille structure.

As may furthermore be seen in FIG. 5, the lowermost part of the interior is formed by the required electronics which supply power to the blower device 61, the UV-C radiation source 50, the sensor or sensors 90 and any wireless (or cabled) transmission units and/or control devices. A connection 12 for the current and voltage supply is likewise shown in FIG. 2, although it is not represented again in FIG. for the sake of simple representation. Alternatively, the current supply may naturally also be carried out using batteries.

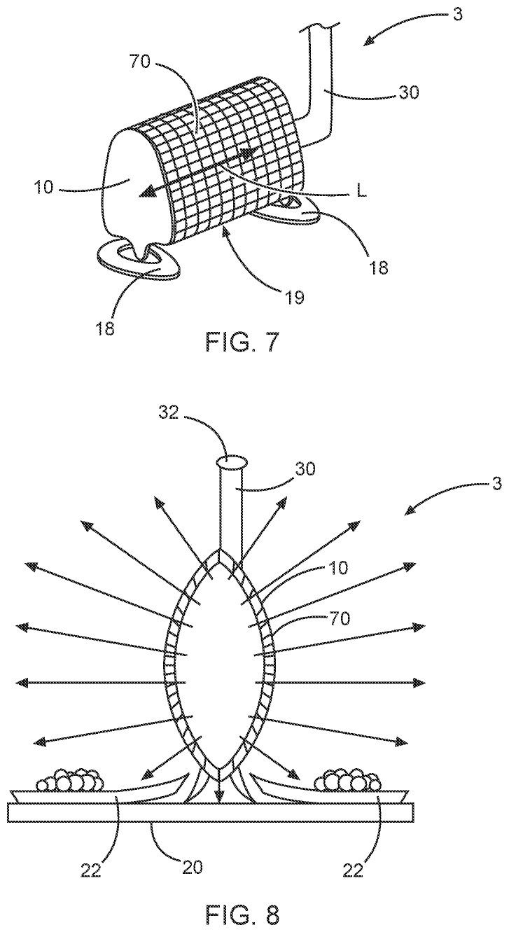

FIGS. 7 to 9 show a third exemplary embodiment with the aid of a further table-top apparatus 3. The table-top apparatus 3 in this case has a housing 10 with housing feet 18, by which the table-top apparatus 3 can stand on the surface of the table 20 as shown in FIG. 8. In this way, on the one hand it is also possible to release an air flow downward, and on the other hand plates 22 or bowls may also still be placed underneath the table-top apparatus 3 on very small tables 20 (see FIG. 8).

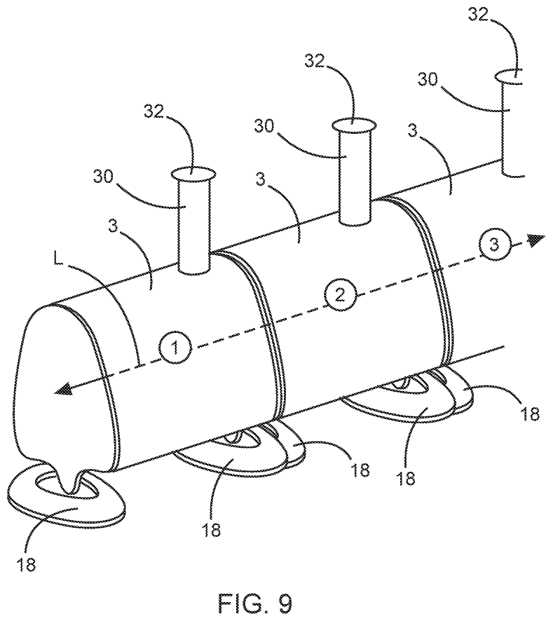

With the aid of docking means, for example on the apparatus feet 18 as well, a plurality of table-top apparatuses 3 may respectively be fastened to one another on the end sides in the direction of the longitudinal axis L, as is shown in FIG. 9. They therefore form a row. In this way, relatively long tables or even beer tables may also be equipped over the entire length with table-top apparatuses in order to provide safe spatial zones 5 on both sides. The suction ducts 30 may in this case be guided compactly upward through the interior 14 itself.

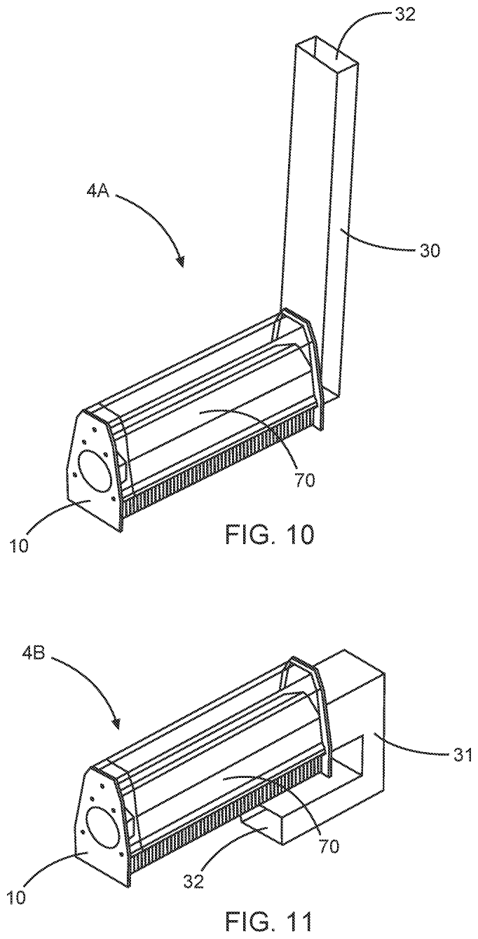

FIGS. 10 and 11 show further exemplary embodiments of table-top apparatuses 4A and 4B. The table-top apparatus 4A of FIG. corresponds substantially to the exemplary embodiments above: the suction duct is fed vertically upward in order to place the opening 32 of the suction duct 30 above the upper boundary of the spatial zone 5. The table-top apparatus 4B of FIG. 11, on the other hand, has a suction duct 31 which is guided horizontally along the table surface and is then bent around the table edge (not shown) so that its opening 32 is positioned below a tabletop of the table 20. Since the spatial zone 5 may often scarcely extend this far, germ-laden air 120 for disinfection may also thus be taken in from below the table.

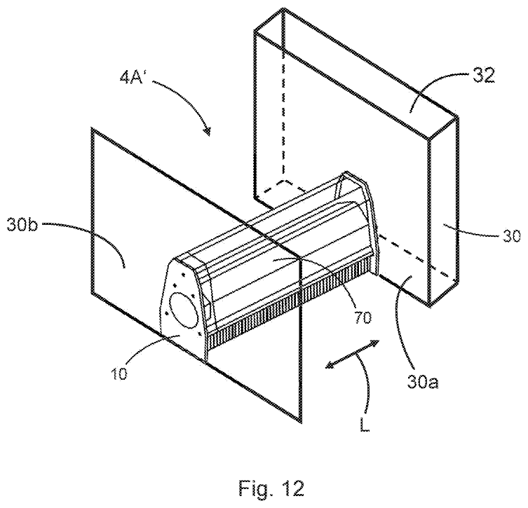

FIG. 12 shows a further exemplary embodiment of a table-top apparatus 4A′, which corresponds substantially to the embodiment in FIG. 10 but is provided with lateral droplet protection 30a, 30b. The lateral droplet protection 30a, 30b protects inter alia the air outlet device 70 and for example the persons sitting at this table-top apparatus 4A′ (cf. FIG. 1) from other persons located to the side of them. For this purpose, on the one hand the suction duct 30 is modified in such a way that it extends perpendicularly to the longitudinal axis L of the housing 10, with an extent corresponding to a first droplet protection shield 30a. The suction duct 30 in this embodiment thus has the extra function of a droplet protection shield 30a. At the opposite end side of the housing 10, on the other hand, a further lateral droplet protection shield 30b which extends in the plane of this end side, that is to say perpendicularly to the longitudinal axis L of the housing 10, is provided. The droplet protection 30a, 30b is for example formed so as to be transparent, for example from plexiglass or the like. The droplet protection shields 30a, 30b may for example extend over the entire width of the table and may be suitably high, according to requirements.

Laminar air flows have been described as being advantageous in the exemplary embodiments above. They are not, however, absolutely necessary in order to generate the spatial zone 5 described, and other flow profiles are also possible according to further exemplary embodiments.

Furthermore, the spatial zone has been described as being approximately spherical (in planes transverse to the longitudinal axis) in the exemplary embodiments above. For example, it is nevertheless also possible to obtain other cross sections in relation to the longitudinal axis, for example ones in which spatial zone regions that are connected by indented regions (above the table-top apparatus) are formed on the left and right of the table-top apparatus. In such cases, the suction duct may also be configured to be significantly shorter, for example with lengths d of 20-60 cm.

Furthermore, it is also possible to obtain fresh air for the UV disinfection through a duct system from outside the restaurant. The present concept of the enclosing spatial zone is not affected by this.

### LIST OF REFERENCES

table-top apparatus 1, 2, 3, 4
germ-inactivated spatial zone 5
housing 10
electrical connection, power supply 12
electronics 13
interior 14
bottom 15
air repository 16
holes to the interior 17
apparatus feet 18
space under the table-top apparatus 19
table 20
plate 22
suction duct 30
droplet protection shield 30a, 30b
opening 32
UV-C radiation source, mercury low-pressure lamp 50
reflector 52
blower device 60
tube (of the blower device or air repository) 62
air outlet device 70
inner first grille structure 71
outer second grille structure 72
membrane 74
air outlet openings 76, 77
sensor 90
persons, users, guests (restaurant, café) 101, 102
unsterilized air to be suctioned 120
suctioned air 121
sterilized air 122
air flowing out 123
heads of the persons, users, guests 201, 202

1. A table-top apparatus for generating a substantially germ-inactivated spatial zone (5), comprising:
a housing with an interior which has a longitudinal axis;
a blower device, which is configured to take air in from outside the housing and convey the air into the interior of the housing;
a radiation source, which is configured to emit light in a UV-C spectral range into the interior in order to inactivate or kill germs in the air taken in to produce sterilized air; and
an air outlet device, through which the sterilized air can flow out from the interior into a space surrounding the housing,
wherein the air outlet device is configured to produce a low-turbulence, or laminar flow in the sterilized air flowing out, which assists formation of the substantially germ-inactivated spatial zone within the space surrounding the housing, and
wherein the substantially germ-inactivated spatial zone thereby generated is closed off and fully encloses the housing, when the housing is observed in a plane perpendicular to the longitudinal axis of the housing.

2. The table-top apparatus as claimed in claim 1, wherein
an opening is provided, through which the air can be taken in outside the substantially germ-inactivated spatial zone and conveyed into the interior of the housing, the blower device being configured to drive an intake of the air through the opening.

3. The table-top apparatus as claimed in claim 2, wherein
the substantially germ-inactivated spatial zone has a maximum extent from the housing,
a suction duct is provided, which has the opening through which the air to be taken in is taken in, and
a distance of the opening from the housing is greater than the maximum extent of the substantially germ-inactivated spatial zone as calculated from the housing.

4. The table-top apparatus as claimed in claim 3, wherein
the distance of the opening of the suction duct from the housing is 80 cm or more, or 90 cm or more, or 100 cm or more.

5. The table-top apparatus as claimed in claim 3, wherein
the maximum extent of the substantially germ-inactivated spatial zone as calculated from the housing is 80 cm or less, or 70 cm or less, or 60 cm or less.

6. The table-top apparatus as claimed in claim 1, wherein
the air outlet device has a grille structure or a pattern of holes having a multiplicity of air outlet openings, each of which gives rise to flow vectors in the sterilized air flowing out through them, the flow vectors in their entirety covering a full semicircle of at least 180 degrees in planes perpendicular to the longitudinal axis, perpendicularly over a surface on which the table-top apparatus is put during operation.

7. The table-top apparatus as claimed in claim 1, wherein
the air outlet device is configured in such a way that two laminar flows substantially lying opposite one another are formed perpendicularly to the longitudinal axis, a flow rate in each case being 0.5 m/s or less.

8. The table-top apparatus as claimed in claim 7, wherein
the flow rate is in each case 0.2 m/s or less, or about 0.1 m/s.

9. The table-top apparatus as claimed in claim 1, wherein
the radiation source is configured to emit light in the UV-C spectral range with a dose of 50 J/m² or more, or 100 J/m² or more.

10. The table-top apparatus as claimed in claim 1, furthermore comprising
a reflector device, by which the interior is irradiated with radiation emitted by the radiation source.

11. The table-top apparatus as claimed in claim 1, wherein
the interior is mirrored in order to achieve homogeneous illumination of the interior.

12. The table-top apparatus as claimed in claim 1, wherein
the air outlet device has an inner first grille structure, an outer second grille structure, and an air-permeable membrane arranged between them.

13. The table-top apparatus as claimed in claim 12, wherein
the outer second grille structure and the air-permeable membrane are configured so as to be mechanically replaceable by using manually releasable fastening means.

14. The table-top apparatus as claimed in claim 1, furthermore comprising
a sensor for recording data relating to a function and an operating state of the table-top apparatus, including a dose of the light emitted, an air flow generated or a distance of a person located in the space surrounding the housing.

15. The table-top apparatus as claimed in claim 14, furthermore comprising
a monitoring unit having a transmission and reception unit, which is configured to communicate such data relating to the function and the operating state of the table-top apparatus to an external control device.

16. The table-top apparatus as claimed in claim 1, furthermore comprising
docking means, by which a further table-top apparatus of a same design may be docked to the table-top apparatus along the longitudinal axis, in order to increase the substantially germ-inactivated spatial zone in a direction of the longitudinal axis.

17. The table-top apparatus as claimed in claim 1, furthermore comprising
at least one droplet protection shield which extends at least on an end side of the housing and in the plane perpendicular to the longitudinal axis of the housing.
